(19) United States
(12) Patent Application Publication
(10) Pub. No.: US 2017/0252281 A1
(43) Pub. Date: Sep. 7, 2017

(54) USE OF SPECIFIC OLIGOPEPTIDES FOR STIMULATING THE NATURAL PIGMENTATION PROCESS IN CUTANEOUS APPENDAGES

(71) Applicant: Henkel AG & Co. KGaA, Duesseldorf (DE)

(72) Inventors: Melanie Giesen, Geldern (DE); Guido Fuhrmann, Erkelenz (DE)

(73) Assignee: Henkel AG & Co. KGaA, Duesseldorf (DE)

(21) Appl. No.: 15/396,711

(22) Filed: Jan. 2, 2017

Related U.S. Application Data
(63) Continuation of application No. PCT/EP2015/060870, filed on May 18, 2015.

(30) Foreign Application Priority Data
Jul. 3, 2014 (DE) 10 2014 212 921.4

(51) Int. Cl. A61K 8/64; A61Q 5/06

(52) U.S. Cl. CPC A61K 8/64; A61Q 5/065; A61K 2800/70

(57) ABSTRACT

Specific oligopeptides, and a cosmetic agents that include such oligopeptides, are used for stimulating the natural pigmentation process in cutaneous appendages, in particular human hair and hair follicles.

### FIELD OF THE INVENTION

The present invention generally relates a cosmetic, non-therapeutic use of at least one specific oligopeptide for stimulating the natural pigmentation process in cutaneous appendages, in particular in human hair and hair follicles.

The present invention also relates to a cosmetic, non-therapeutic use of a cosmetic agent, which includes at least one specific oligopeptide, for stimulating the natural pigmentation process in cutaneous appendages, in particular in human hair and hair follicles.

The present invention additionally relates to a cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, in which a specific oligopeptide is applied to cutaneous appendages, in particular human hair and hair follicles, and is rinsed out after a given leave-in time.

Finally, the present invention relates to a cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, in which a cosmetic agent, including at least one specific oligopeptide, is applied to cutaneous appendages, in particular human hair and hair follicles, and is rinsed out after a given leave-in time.

### BACKGROUND OF THE INVENTION

Besides its actual physiological functions of providing heat insulation and protecting against light, hair has a psychosocial function that must not be underestimated. Among other things, it is a means of communicating with others and is a sign of a person's individuality. Changes such as graying for example may have an adverse effect on the self-confidence of the person in question. Consumers therefore have the desire to color hair that has turned gray.

To this end, the hair can be treated, for example using chemical colorants which, due to their chemically aggressive nature, may lack tolerability (itching, burning, stinging) and may lead to damage to the hair in the event of long-term use. In addition, when using such colorants, the roots of the hair must be regularly colored thereafter in order to ensure a uniform hair color.

Also known in the prior art are products in which biological products are used as dyes for covering gray. However, these products do not always perform satisfactorily.

In order to avoid the disadvantages mentioned above, it therefore appears to be desirable to influence the graying process directly at the roots of the hair and/or in the hair follicles in order in this way to achieve a repigmentation of human hair and/or a reduction or even a resolving of the hair graying phenomenon.

Pigmentation in the hair follicle takes place through the synthesis of melanin, a pigment which is produced and stored in the melanosomes of the melanocytes. Melanin is responsible for the natural color of the hair, the hair color being controlled by the proportions of two different types of melanin, eumelanin and pheomelanin. The synthesis of melanin, which is also known as melanogenesis, is controlled by a defined complex set of molecular signals. In melanogenesis, first tyrosine is converted by the enzyme tyrosinase into L-dihydroxyphenylalanine (also known as L-DOPA), which is then converted via a plurality of further intermediate stages into different melanin pigments. The influencing or disruption of the melanogenesis in gray hair follicles may be caused for example by a modification of the function of the molecular signals. One consequence of this modification is the reduction in melanin synthesis, which leads to graying of the follicle. The complex set of molecular signals which influence melanogenesis includes inter alia the expression of MCR1 (melanocortin receptor 1), gp100 and also c-kit. MCR1 and c-kit are receptors which transmit key signals of melanogenesis by the binding of their ligands alphamelanocyte stimulating hormone and stem cell factor in the cell interior. Gp100 is a protein of the melanosome membrane and additionally regulates further proteins relevant to melanogenesis. Since these parameters are of essential importance to hair follicle pigmentation, by influencing these signals it is possible to maintain or even reactivate the synthesis of melanin in the hair follicle cells. By using suitable active substance formulations, it is therefore possible to retain the pigmentation and thus the youthfulness of the hair.

Active substances which lead to a stimulation of the pigmentation of skin and hair are already known from the prior art. For instance, laid-open specification WO 91/07945 A1 describes the use of xanthines as active substances. In addition, the use of carnitine or derivatives thereof in combination with purine or derivatives thereof for the pigmentation of human hair is known from laid-open specification DE 102009044964 A1.

In the prior art, however, there is still a need to provide highly potent active substances or active substance combinations which influence melanogenesis in the hair follicle and lead to a repigmentation of human hair.

It is therefore desirable to provide active substances which avoid or alleviate the disadvantages of the prior art and which lead to a significant influencing of the natural pigmentation process, in particular in the hair or in the hair follicle. Furthermore, these active substances should be able to be easily incorporated in cosmetic compositions without leading to stability problems or incompatibilities with other ingredients.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic, non-therapeutic use of at least one oligopeptide which comprises at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)

in which A, B, C, D, E and F, each independently of one another, represent the amino acids Leu, Tyr, lle, Arg, Val, Ser, Thr and Asp; a, b, c, d, e and f, each independently of one another, represent the integers 0 or 1; and the amino group is in non-protonated and/or protonated form and the carboxy groups are in non-deprotonated and/or deprotonated form, for stimulating the natural pigmentation process in cutaneous appendages.

A cosmetic, non-therapeutic use of a cosmetic agent for stimulating the natural pigmentation process in cutaneous appendages, wherein the cosmetic agent includes, in a cosmetically acceptable carrier, at least one oligopeptide comprising at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)

in which A, B, C, D, E and F, each independently of one another, represent the amino acids Leu, Tyr, lle, Arg, Val, Ser, Thr and Asp; a, b, c, d, e and f, each independently of one another, represent the integers 0 or 1; and the amino group is in non-protonated and/or protonated form and the carboxy groups are in non-deprotonated and/or deprotonated form.

A cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, which comprises the following method steps: a) providing an oligopeptide which includes at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)

in which A, B, C, D, E and F, each independently of one another, represent the amino acids Leu, Tyr, lle, Arg, Val, Ser, Thr and Asp; a, b, c, d, e and f, each independently of one another, represent the integers 0 or 1; and the amino group is in non-protonated and/or protonated form and the carboxy groups are in non-deprotonated and/or deprotonated form; b) topically applying the at least one oligopeptide provided in method step a) to cutaneous appendages, in particular hair; c) leaving the at least one oligopeptide applied in method step b) on the cutaneous appendages, in particular hair, for a duration of from 30 seconds to 2 hours; and d) optionally rinsing out the cosmetic agent that was left on the cutaneous appendages, in particular the hair, in method step c).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that the use of at least one specific oligopeptide leads to a stimulation of the natural pigmentation process in cutaneous appendages, such as human hair and hair follicles, and in particular to a significantly increased synthesis of melanin in the cutaneous appendages, and in this way an excellent repigmentation of human hair and/or a prevention of the graying of human hair can be achieved.

A first subject matter of the present invention is a cosmetic, non-therapeutic use of at least one oligopeptide which comprises at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1,
the amino group is in non-protonated and/or protonated form and the carboxy groups are in non-deprotonated and/or deprotonated form,
   for stimulating the natural pigmentation process in cutaneous appendages.

The use of the aforementioned specific oligopeptide leads to a stimulation of the natural pigmentation process in cutaneous appendages, in particular in human hair and hair follicles, which manifests itself in a significantly increased synthesis of melanin in the cutaneous appendages. Moreover, these specific oligopeptides can be easily incorporated in cosmetic agents for repigmenting human hair and do not lead to incompatibilities or intolerances with the ingredients used in said agents, so that a high degree of storage stability of the cosmetic agents is ensured when using the specific oligopeptides described above.

In the context of the present invention, the term “oligopeptide” will be understood to mean condensation products of amino acids which are linked in an acid amide-type manner by peptide bonds and comprise 3 to 25 amino acids.

Furthermore, in the context of the present invention, the term “stimulation of the natural pigmentation process” will be understood to mean the positive influencing of the aging-induced decline in the natural color/coloring/pigmentation of cutaneous appendages, in particular the positive influencing of the aging-induced decline in the natural, that is to say biological, pigmentation process in cutaneous appendages. However, it does not encompass the positive influencing of illness-induced decline of the natural color/coloring/pigmentation of cutaneous appendages.

Furthermore, in the context of the present invention, the term “cutaneous appendages” describes hair and hair follicles, such as body hair, beard hair and head hair, preferably beard hair and head hair, in particular head hair and the corresponding hair follicles.

In the context of the present invention, the amino acids included in the oligopeptide used according to the invention will be shortened to the so-called 3-letter code. The amino acids used in the context of the present invention, as well as their shortened form in the 3-letter code, are listed below:

Glu=glutamic acid; Leu=leucine; Tyr=tyrosine; lle=isoleucine; Arg=arginine; Val=valine; Ser=serine; Thr=threonine; Asp=aspartic acid.

In the above formula (I) and in all the formulae below, the bracketed hydrogen atom of the amino group, like the bracketed hydroxyl group of the acid function, means that the groups in question may be present as such (in which case it is an oligopeptide having the respective number of amino acids as shown for example in the above formula (I)). In this case, the amino group may be free or protonated and the carboxy groups may be free or deprotonated. However, in the context of the present invention, it is also possible that the amino acid sequence is present in an oligopeptide which also comprises further amino acids. In this case, the bracketed constituent(s) of the above formula (I) and of all the formulae below are replaced by the further amino acid residue(s), the amino groups of which may be free or protonated and the carboxy groups of which may be free or deprotonated.

According to one preferred embodiment of the present invention, the natural pigmentation process of the hair is stimulated by the use according to the invention of the at least one specific oligopeptide. In the context of the present invention, the term “hair” is to be understood to mean both keratin fibers as such and also the hair follicles, in particular human keratin fibers and hair follicles. Preferably, this term denotes human head hair and beard hair and also the hair follicles thereof.

In the context of the present invention, it is particularly preferred if at least one sub-step of the natural pigmentation process is stimulated by the use according to the invention of the at least one specific oligopeptide. This stimulation relates in particular to the regulation of those molecular signals which influence the biological or natural pigmentation process. Preferably, the regulation of the biological or natural pigmentation process is through gene regulation, such as for example regulation on the expression level, and/or through enzyme regulation, such as for example regulation on the activity level, and/or through regulation on a hormone level. In the context of this embodiment, the stimulation of a sub-step of the natural pigmentation process may take place for example through gene regulation or enzyme regulation of tyrosinase.

Furthermore, in the context of one embodiment of the present invention, it is preferred that the pigmentation of the hair is stimulated and/or improved by the use according to the invention of the at least one specific oligopeptide. In the context of the present invention, the term “stimulation and/or improvement” will be understood to mean in particular the improvement, increase and/or stimulation of the transportation of the melanosomes into the keratinocytes surrounding the hair follicles, and also the pigmentation, perceptible by eye or by suitable measurement methods, of the individual hair, of a selection of hairs, in particular an area of hair-covered skin, in particular the scalp, or of all of the head hair and/or beard hair.

According to one particularly preferred embodiment of the present invention, melanogenesis, in particular in the hair follicle, is stimulated by the use according to the invention of the at least one specific oligopeptide. According to the invention, stimulation of melanogenesis, preferably of melanogenesis in the hair follicle, is with particular preference to be understood to mean the stimulation, increase, inducement or improvement of melanin synthesis in the melanocytes (preferably the melanocytes in the hair follicle). This is achieved for example by increasing the gene expression of signal molecules such as MCR1 (melanocortin receptor 1), gp100 and c-kit. In particular, melanogenesis in the hair or hair follicles of the hair-covered scalp and/or of the beard, in particular of the hair-covered scalp and/or the beard of humans, is stimulated.

In the context of the present invention, it is preferred if graying of the hair is prevented and/or reduced by the use according to the invention of the at least one specific oligopeptide. In the context of the present invention, the term “graying of the hair” is to be understood to mean the graying of the hair that is visually perceptible through the mixing of white and pigmented hair, the dilution of the pigment in an individual hair, and the graying of an individual hair through the natural aging process. However, it does not encompass pathological or illness-induced graying of the hair.

Prevention of hair graying takes place in particular on hair that has not yet turned gray. In contrast, a reduction of the graying of hair can take place both on hair that has already turned gray and on hair that has not yet turned gray. In the case of hair that has already turned gray, hair follicles in which melanogenesis does not function, no longer functions or does not entirely function or has been disrupted or reduced are induced or stimulated again for melanogenesis to take place, whereas in hair/hair follicles that have not yet turned gray a disruption, reduction or downregulation of melanogenesis is prevented or alleviated.

According to another preferred embodiment of the present invention, it may be provided that hair that has turned gray is repigmented by the use according to the invention of the at least one specific oligopeptide.

In the context of the present invention, it is preferred if the use takes place topically. According to the invention, a topical application will be understood to mean the application to the cutaneous appendages, in particular the scalp and the skin of the face.

It is preferred if the at least one oligopeptide used according to the invention comprises 5 to 15 amino acids, preferably 6 to 13 amino acids, more preferably 7 to 12 amino acids, in particular 8, 9 or 10 amino acids.

The molecular weight of the oligopeptide used according to the invention may vary depending on whether further amino acids are bound to the amino acid sequence Glu-Glu-Glu and depending on the type of these amino acids. According to the invention, preference is given to the use of at least one oligopeptide which has an average molecular weight M_{w} of 400 to 2000 Da, preferably 450 to 1500 Da, more preferably 500 to 1200 Da, in particular 550 to 1000 Da. The average molecular weight M_{w} of the at least one oligopeptide can be determined for example by gel permeation chromatography (GPC) using polystyrene as the internal standard according to DIN 55672-3.

According to one particularly preferred embodiment of the present invention, the at least one oligopeptide has a tyrosine content of less than 6%, preferably less than 5%, more preferably less than 4%, in particular less than 3%. Despite the low tyrosine content, which serves as a starting substance in melanin synthesis, the use of at least one oligopeptide leads to an excellent stimulation of the natural pigmentation process in the form of an increase in melanin synthesis and thus to a particularly effective repigmentation of hair that has turned gray. The tyrosine content of the oligopeptide used according to the invention can be determined by various analysis methods, such as Edman degradation (P. Edman, A method for the determination of amino acid sequence in peptides; Arch. Biochem. 22, 1949, page 475) or by means of HPLC methods (Cohen et al., “Compositional Protein Analysis using 6-Aminoquinolyl-N-Hydroxasuccinimyl Carbamate, a Novel Derivatization Reagent”, Techniques in Protein Chemistry IV, 1993, pages 289 to 298).

As can be seen from formula (I) above, the at least one oligopeptide used according to the invention does not consist solely of the peptide sequence Glu-Glu-Glu, but rather has further amino acids bound to said sequence. The use according to the invention of at least one specific oligopeptide leads to a significant stimulation of melanin synthesis and thus to a reduction or prevention of graying of the hair. Oligopeptides which are used with particular preference, and amino acid sequences included in the preferred oligopeptides, will be described below:

One oligopeptide which is used with preference is characterized in that, in the amino acid sequence of formula (I), B represents the amino acid Tyr, a, c, d, e and f each represent the integer 0, and b represents the integer 1. Oligopeptides in the context of this embodiment therefore include the amino acid sequence (H)NH-Tyr-Glu-Glu-Glu-CO(OH).

Another oligopeptide which is used with preference is characterized in that, in the amino acid sequence of formula (I), C represents the amino acid lle, a, b, d, e and f each represent the integer 0, and c represents the integer 1. The amino acid sequence of oligopeptides in the context of this embodiment is therefore (H)NH-Glu-Glu-Glu-Ile-CO(OH).

In addition, another oligopeptide which is used with preference is characterized in that, in the amino acid sequence of formula (I), B represents the amino acid Tyr, C represents the amino acid lle, a, d, e and f each represent the integer 0, and b and c each represent the integer 1. Oligopeptides in the context of this embodiment therefore include the amino acid sequence (H)NH-Tyr-Glu-Glu-Glu-Ile-CO(OH).

Moreover, another oligopeptide which is used additionally includes the amino acid arginine (Arg), which is bound via its amino function to the amino acid isoleucine (lle). It is therefore particularly preferred in the context of the present invention if, in the amino acid sequence of formula (I), B represents the amino acid Tyr, C represents the amino acid lle, D represents the amino acid Arg, a, e and f each represent the integer 0, and b, c and d each represent the integer 1. The oligopeptide included in the cosmetic agents used according to the invention in this embodiment therefore includes the amino acid sequence (H)NH-Tyr-Glu-Glu-Glu-Ile-Arg-CO(OH).

In addition, it may be provided in the context of the present invention that one oligopeptide which is used with preference additionally has the amino acid valine (Val), which is bound via its amino function to the amino acid arginine (Arg). It is therefore particularly preferred in the context of the present invention if, in the amino acid sequence of formula (I), B represents the amino acid Tyr, C represents the amino acid lle, D represents the amino acid Arg, E represents the amino acid Val, a and f each represent the integer 0, and b, c, d and e each independently represent the integer 1. The oligopeptide included in the cosmetic agents used according to the invention in this embodiment therefore includes the amino acid sequence (H)NH-Tyr-Glu-Glu-Glu-Ile-Arg-Val-CO(OH).

According to one particularly preferred embodiment of the present invention, the oligopeptide used additionally includes the amino acid leucine (Leu), which is bound via its amino function to the amino acid valine (Val). It is therefore particularly preferred in the context of the present invention if, in formula (I), B represents the amino acid Tyr, C represents the amino acid lle, D represents the amino acid Arg, E represents the amino acid Val, F represents the amino acid Leu, a represents the integer 0, and b, c, d, e and f each represent the integer 1. The oligopeptide included in the cosmetic agents used according to the invention in this very particularly preferred embodiment therefore includes the amino acid sequence (H)NH-Tyr-Glu-Glu-Glu-Ile-Arg-Val-Leu-CO(OH).

According to another particularly preferred embodiment of the present invention, the oligopeptide used additionally includes the amino acid leucine (Leu), which is bound via its carboxylic acid function to the amino acid tyrosine (Tyr). It is therefore particularly preferred in the context of the present invention if, in the amino acid sequence of formula (I), A represents the amino acid Leu, B represents the amino acid Tyr, C represents the amino acid lle, D represents the amino acid Arg, E represents the amino acid Val, F represents the amino acid Leu, and a, b, c, d and e each represent the integer 1. The oligopeptide included in the cosmetic agents used according to the invention in this very particularly preferred embodiment therefore includes the amino acid sequence (H)NH-Leu-Tyr-Glu-Glu-Glu-Ile-Arg-Val-Leu-CO(OH).

However, it is also possible in the context of the present invention to use two mutually different oligopeptides which satisfy the criteria mentioned above but which differ from one another in at least one amino acid. It may therefore be preferred according to the invention if two mutually different oligopeptides are used, wherein
i) the first oligopeptide a) comprises at least one amino acid sequence of formula (I) in which A represents the amino acid Leu, B represents the amino acid Tyr, C represents the amino acid lle, D represents the amino acid Arg, E represents the amino acid Val and F represents the amino acid Leu, and a, b, c, d, e and f each represent the integer 1, and
ii) the second oligopeptide b) comprises at least one amino acid sequence of formula (I) in which B represents the amino acid Tyr, C represents the amino acid lle, D represents the amino acid Arg, E represents the amino acid Val and F represents the amino acid Leu, a represents the integer 0, and b, c, d, e and f each represent the integer 1.

In the context of this embodiment, it is particularly preferred if the oligopeptide a) has the amino acid sequence (H)NH-Leu-Tyr-Glu-Glu-Glu-Ile-Arg-Val-Leu-CO(OH) and the oligopeptide b) has the amino acid sequence (H)NH-Tyr-Glu-Glu-Glu-Ile-Arg-Val-Leu-CO(OH). The use according to the invention of two mutually different oligopeptides a) and b) leads to an excellent stimulation of the natural pigmentation process in the form of melanin synthesis and thus to a significant increase in the melanin content in the hair follicles. By increasing the melanin content, the graying of the hair can be avoided or reduced or even resolved.

The oligopeptides which are used in the context of the present invention and which satisfy the conditions mentioned above can advantageously be obtained from keratin materials. It is preferred according to the invention that these oligopeptides are present in high proportions relative to the total keratin peptide content. It is therefore very particularly preferred that the highest possible proportion of all keratin oligopeptides used according to the invention satisfies the conditions mentioned above.

According to one preferred embodiment of the present invention, therefore, use is made of oligopeptides in which at least 0.1% by weight, preferably at least 0.5% by weight, particularly preferably at least 1% by weight, more preferably at least 2.5% by weight, yet more preferably at least 5% by weight and in particular at least 10% by weight of all oligopeptides have an amino acid sequence of formula (I) in which B represents the amino acid Tyr, C represents the amino acid lle, D represents the amino acid Arg, E represents the amino acid Val and F represents the amino acid Leu, a represents the integer 0, and b, c, d, e and f each represent the integer 1. The conditions mentioned above concern the total content of peptides in the oligopeptides used according to the invention, which stem from keratin materials and have the amino acid sequence of form (H)NH-Tyr-Glu-Glu-Glu-Ile-Arg-Val-Leu-CO(OH).

With preference according to the invention, the at least one oligopeptide is used in a total amount of 0.001 to 3% by weight, preferably 0.002 to 2.8% by weight, more preferably 0.005 to 2.5% by weight, yet more preferably 0.008 to 2.3% by weight, in particular 0.01 to 2.0% by weight, based on the total weight of a composition including the at least one oligopeptide, optionally a carrier and optionally further ingredients. The use of the at least one oligopeptide in the aforementioned amounts results in a significant increase in melanin synthesis by stimulating the natural pigmentation process. In addition, no negative interactions with any further ingredients used occurs when using these amounts of the at least one oligopeptide, so that the compositions have a high degree of stability despite the use of the at least one oligopeptide.

The aforementioned total amount of the at least one oligopeptide refers here to a composition which comprises, besides the at least one oligopeptide, preferably at least one cosmetically acceptable carrier and optionally further ingredients customary in cosmetic compositions.

Another subject matter of the present invention is a cosmetic, non-therapeutic use of a cosmetic agent for stimulating the natural pigmentation process in cutaneous appendages, wherein the cosmetic agent includes, in a cosmetically acceptable carrier, at least one oligopeptide comprising at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1,
the amino group is in non-protonated and/or protonated form and the carboxy groups are in non-deprotonated and/or deprotonated form.

The use of cosmetic agents including specific oligopeptides leads to a stimulation of the natural pigmentation process in cutaneous appendages, in particular in human hair and/or hair follicles, in the form of an increased synthesis of the natural hair pigment melanin. In this way, graying of the hair is effectively reduced or even avoided.

The cosmetic agents used according to the invention are preferably in the form of hair treatment agents. Hair treatment agents in the context of the present invention are for example hair coloring agents, bleaching agents, hair shampoos, hair conditioners, conditioning shampoos, hairsprays, rinse-out hair conditioners, intensive hair conditioners, hair masks, hair tonics, perm fixing solutions, hair coloring shampoos, hair coloring agents, hair setting agents, hair smoothing agents, hair styling preparations, blow-drying lotions, mousses, hair gels, hair waxes or combinations thereof. Particularly preferred hair treatment agents are characterized in that they are in the form of a shampoo, hair tonic, intensive hair conditioner, rinse-out hair conditioner, hair mouse, hair setting agent, hairspray, hair gel and/or hair coloring agent. In view of the fact that consumers are often reluctant to use multiple different agents and/or multiple application steps, preference is given to using according to the invention those cosmetic agents which the consumer already uses. Cosmetic agents used with preference are therefore shampoos, conditioners or hair tonics.

The agents used according to the invention include a cosmetic carrier. According to the invention, the cosmetic carrier is preferably aqueous, alcoholic or aqueous-alcoholic.

In the context of the invention, an aqueous carrier includes at least 30% by weight, in particular at least 50% by weight water, based on the total weight of the cosmetic agent.

In the context of the present invention, aqueous-alcoholic carriers are to be understood to be water-containing compositions including a C₁-C₄ alcohol in a total amount of 3 to 90% by weight, based on the total weight of the cosmetic agent, in particular ethanol and/or isopropanol.

The agents according to the invention may additionally include further organic solvents, such as for example methoxybutanol, ethyl diglycol, 1,2-propylene glycol, n-propanol, n-butanol, n-butylene glycol, glycerol, diethylene glycol monoethyl ether and diethylene glycol mono-n-butyl ether. Preference is given to all water-soluble organic solvents, the solvent being included in a total amount of 0.1 to 30% by weight, preferably 1 to 20% by weight, in particular 2 to 10% by weight, based on the total weight of the cosmetic agent.

The cosmetic agents used according to the invention may additionally include at least one conditioning substance, selected from the group consisting of cationic polymers, cationic surfactants, silicones, fatty substances and mixtures thereof. According to the invention, the term “conditioning substances” is to be understood to mean those substances which absorb onto keratin materials, in particular onto the hair, and improve the physical and sensory properties both of the hair and also of the product as such. Conditioning agents smooth the uppermost layer of the hair and make it soft and supple.

The cationic polymers which can be used as conditioning substances in the context of the present invention have in the main chain and/or side chain a group which may be “temporarily” or “permanently” cationic. According to the invention, the term “permanently cationic” denotes those polymers which have a cationic group regardless of the pH of the agent. These are usually polymers which include a quaternary nitrogen atom, for example in the form of an ammonium group. Preferred cationic groups are quaternary ammonium groups. In particular, those polymers in which the quaternary ammonium group is bound via a C₁₋₄ hydrocarbon group to a polymer main chain constructed from acrylic acid, methacrylic acid or derivatives thereof have proven to be particularly suitable.

Preferred cationic polymers are selected from the group consisting of poly(methacryloyloxyethyltrimethylammonium chloride) (INCI: Polyquaternium-37), quaternized cellulose derivatives (INCI: Polyquaternium-10), cationic alkyl polyglycosides, cationic honey, cationic guar derivatives, polymeric dimethyldiallylammonium salts and copolymers thereof with esters and amides of acrylic acid and methacrylic acid, copolymers of vinylpyrrolidone with quaternized derivatives of dialkylaminoalkyl acrylate and methacrylate, vinylpyrrolidone/vinylimidazolium methochloride copolymers, quaternized polyvinyl alcohol, Polyquaternium-2, Polyquaternium-7, Polyquaternium-17, Polyquaternium-18, Polyquaternium-24, Polyquaternium-27, and mixtures thereof.

It may be preferred in the context of the present invention if the cationic polymer(s) are included in a total amount of 0.05 to 7.5% by weight, preferably 0.1 to 5% by weight, more preferably 0.2 to 3.5% by weight, in particular 0.25 to 2.5% by weight, based on the total weight of the cosmetic agent used according to the invention.

Also suitable as conditioning substances in the context of the present invention are cationic surfactants from the group consisting of quaternary ammonium compounds and/or esterquats and/or amidoamines.

Cationic surfactants which are used with preference are selected from the group consisting of alkyltrimethylammonium chlorides with preferably 10 to 18 carbon atoms in the alkyl radical, dialkyldimethylammonium chlorides with preferably 10 to 18 carbon atoms in the alkyl radical, trialkylmethylammonium chlorides with preferably 10 to 18 carbon atoms in the alkyl radical, cetyltrimethylammonium chloride, stearyltrimethylammonium chloride, distearyldimethylammonium chloride, lauryldimethylammonium chloride, lauryldimethylbenzylammonium chloride, tricetylmethylammonium chloride, Quaternium-27, Quaternium-83, N-methyl-N(2-hydroxyethyl)-N,N-(ditalgacyloxyethyl)ammonium methosulfate, N-methyl-N(2-hydroxyethyl)-N,N-(distearoyloxyethyl)ammonium methosulfate, N,N-dimethyl-N,N-distearoyloxyethylammonium chloride, N,N-di-(2-hydroxyethyl)-N,N-(fatty acid ester ethyl)ammonium chloride, and mixtures thereof.

The cationic surfactant(s) are included in the cosmetic agents used according to the invention preferably in a total amount of 0.5 to 50% by weight, more preferably 1 to 40% by weight, particularly preferably 1.5 to 30% by weight, in particular 2 to 20% by weight, based on the total weight of the cosmetic agent.

Furthermore, it may be preferred in the context of the present invention if the cosmetic agents used according to the invention include as a conditioning substance at least one silicone which is selected from the group consisting of
(i) polyalkylsiloxanes, polyarylsiloxanes, polyalkylarylsiloxanes, which are volatile or non-volatile, straight-chain, branched or cyclic, crosslinked or uncrosslinked;
(ii) polysiloxanes which include in their general structure one or more organofunctional groups selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane (A)/polyoxyalkylene (B) block copolymers of type (A-B)ₙ where n>3;
(iv) grafted silicone polymers having a non-silicone-containing organic backbone, which consist of an organic main chain formed of organic monomers including no silicone, onto which at least one polysiloxane macromer has been grafted in the chain and also optionally at least at one chain end;
(v) grafted silicone polymers having a polysiloxane backbone onto which non-silicone-containing organic monomers have been grafted, which have a polysiloxane main chain onto which at least one organic macromer including no silicone has been grafted in the chain and also optionally at least at one end thereof;
   or mixtures thereof.

Preferred silicones are selected from silicones of formula Si-I

(CH₃)₃Si—[O—Si(CH₃)₂]ₓ—O—Si(CH₃)₃ (Si-I),

in which x represents a number from 0 to 100, preferably from 0 to 50, more preferably from 0 to 20, in particular 0 to 10.

These silicones are called DIMETHICONES according to the INCI nomenclature.

Preferred silicones have viscosities of 0.2 to 2 mm² s⁻¹ at 20° C., particular preference being given to silicones having viscosities of 0.5 to 1 mm² s⁻¹.

Particularly preferred cosmetic agents which are used according to the invention include one or more aminofunctional silicones. Regardless of which aminofunctional silicones are used, preference is given to cosmetic agents used according to the invention which include an aminofunctional silicone having an amine number above 0.25 meq/g, preferably above 0.3 meq/g, in particular above 0.4 meq/g. Cosmetic agents which are preferably used include the aminofunctional silicone(s) in a total amount of 0.01 to 10% by weight, preferably 0.1 to 8% by weight, more preferably 0.25 to 7.5% by weight, in particular 0.5 to 5% by weight, based on the total weight of the cosmetic agent.

The use of the cyclic dimethicones, which are called CYCLOMETHICONES according to the INCI nomenclature, is also preferred according to the invention. In particular, cyclic dimethicones of formula Si-II have proven to be advantageous in the context of the present invention

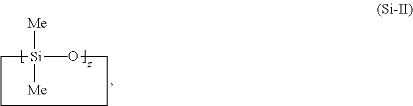

In the above formula (Si-Il), z represents a number from 3 to 200, preferably from 3 to 10, more preferably from 3 to 7, in particular 3, 4, 5 or 6.

Particularly suitable in the context of the present invention is the use according to the invention of cosmetic agents which include the silicone(s) in a total amount of 0.1 to 10% by weight, preferably 0.25 to 7% by weight, in particular 0.5 to 5% by weight, based on the total weight of the cosmetic agent.

The use of fatty substances as conditioning substances is likewise possible in the context of the present invention. Suitable fatty substances are in particular those from the group consisting of plant oils, such as sunflower oil, olive oil, soybean oil, rapeseed oil, almond oil, jojoba oil, orange oil, wheat germ oil, peach kernel oil and the liquid parts of coconut oil, lanolin and derivatives thereof, liquid paraffin oils, isoparaffin oils and synthetic hydrocarbons, di-n-alkyl ethers having a total of 12 to 36 carbon atoms, for example di-n-octyl ether and n-hexyl n-octyl ether, carboxylic acids, particularly linear and/or branched, saturated and/or unsaturated C₈₋₃₀ carboxylic acids, alcohols, particularly saturated, monounsaturated or polyunsaturated, branched or unbranched alcohols having 4 to 30 carbon atoms which may be ethoxylated with 1 to 75, in particular 5 to 20 ethylene oxide units and/or propoxylated with 3 to 30, in particular 9 to 14 propylene oxide units, ester oils, such as esters of C₆₋₃₀ carboxylic acids with C₂₋₃₀ alcohols, hydroxycarboxylic acid alkyl esters, dicarboxylic acid esters, such as di-n-butyl adipate, and diol esters, such as ethylene glycol dioleate or propylene glycol di(2-ethylhexanoate), symmetrical, unsymmetrical or cyclic esters of carbonic acid with alcohols, for example glycerol carbonate or dicaprylyl carbonate (Cetiol® CC), mono-, di- and tricarboxylic acid esters of saturated and/or unsaturated, linear and/or branched carboxylic acids with glycerol, which may be ethoxylated with 1 to 10, in particular 7 to 9 ethylene oxide units, for example PEG-7 Glyceryl Cocoate.

It is preferred in the context of the present invention if the fatty substance(s) are included in the cosmetic agents used according to the invention in a total amount of 0.1 to 10% by weight, preferably 0.25 to 7% by weight, in particular 0.5 to 5% by weight, based on the total weight of the cosmetic agent.

For esthetic reasons, “clear” products are often preferred by consumers. Preferred cosmetic agents used according to the invention are therefore characterized in that they are transparent or translucent. In the context of the present invention, transparent or translucent will be understood to mean a composition which has an NTU value of less than 100. The NTU value (Nephelometry Turbidity Unit; NTU) is a unit used in water treatment for measuring turbidity in liquids. It is the unit of a turbidity of a liquid as measured using a calibrated nephelometer.

With regard to further preferred embodiments of the use according to the invention of the cosmetic agent including at least one specific oligopeptide, what has been stated in relation to the use according to the invention of specific oligopeptides applies mutatis mutandis.

In addition, a further subject matter of the present invention is a cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, which comprises the following method steps:
a) providing an oligopeptide which includes at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1,
the amino group is in non-protonated and/or protonated form and the carboxy groups are in non-deprotonated and/or deprotonated form.
b) topically applying the at least one oligopeptide provided in method step a) to cutaneous appendages, in particular hair,
c) leaving the at least one oligopeptide applied in method step b) on the cutaneous appendages, in particular hair, for a duration of from 30 seconds to 2 hours,
d) optionally rinsing out the cosmetic agent that was left on the cutaneous appendages, in particular the hair, in method step c).

The method according to the invention results in a stimulation of the natural pigmentation process in cutaneous appendages, in particular in human hair and/or hair follicles, in the form of an increased synthesis of the natural hair pigment melanin. In this way, graying of the hair is effectively reduced or even avoided.

According to one preferred embodiment of this subject matter, the cosmetic agent in method step c) is left on the hair for a duration of from 1 minute to 1.5 hours, preferably from 2 minutes to 1 hour, in particular from 3 minutes to 30 minutes.

With regard to further preferred embodiments of the method according to the invention using the specific oligopeptides, what has been stated in relation to the use according to the invention of specific oligopeptides and to the use according to the invention of cosmetic agents including specific oligopeptides applies mutatis mutandis.

Finally, a further subject matter of the present invention is a cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, which comprises the following method steps:
a) providing a cosmetic agent comprising, in a cosmetically acceptable carrier, at least one oligopeptide which includes at least one amino acid sequence of formula (I)

(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1
the amino group is in non-protonated and/or protonated form and the carboxy groups are in non-deprotonated and/or deprotonated form.
b) topically applying the cosmetic agent provided in method step a) to cutaneous appendages, in particular hair,
c) leaving the cosmetic agent applied in method step b) on the cutaneous appendages, in particular hair, for a duration of from 30 seconds to 2 hours,
d) optionally rinsing out the cosmetic agent that was left on the cutaneous appendages, in particular the hair, in method step c).

The method according to the invention results in a stimulation of the natural pigmentation process in cutaneous appendages, in particular in human hair and/or hair follicles, in the form of an increased synthesis of the natural hair pigment melanin. In this way, graying of the hair is effectively reduced or even avoided.

According to one preferred embodiment of this subject matter, the cosmetic agent in method step c) is left on the hair for a duration of from 1 minute to 1.5 hours, preferably from 2 minutes to 1 hour, in particular from 3 minutes to 30 minutes.

With regard to further preferred embodiments of the method according to the invention using cosmetic agents including specific oligopeptides, what has been stated in relation to the use according to the invention of specific oligopeptides, to the use according to the invention of cosmetic agents including specific oligopeptides and to the method according to the invention using specific oligopeptides applies mutatis mutandis.

The following examples explain the present invention without limiting the latter thereto:

### Examples

For analyzing the melanin content, use was made of three-dimensional organotypical hair follicle cultures from dermal papilla cells, hair follicle melanocytes and outer root sheath keratinocytes, which were prepared according to the method described in laid-open specification EP 1455854 A1. These cultures were treated with the compositions E-I, E-II and E-III specified in Table 1 over a period of 7 days (the weights specified in % by weight relate to the total weight of the oligopeptide mixture used, including at least one specific oligopeptide as well as water). Untreated cell cultures were used as a comparison (V).

| Sample | Oligopeptide mixture** [% by weight] |
|---|---|
| V | — |
| E-I* | 0.1 |
| E-II* | 0.5 |
| E-III* | 1.0 |

*according to the invention
**including - based on its total weight - at least 10% by weight of a mixture of oligopeptides consisting of 8 amino acids with the amino acid sequence NH₂-Tyr-Glu-Glu-Glu-Ile-Arg-Val-Leu-COOH and oligopeptides consisting of 9 amino acids with the amino acid sequence NH₂-Leu-Tyr-Glu-Glu-Glu-Ile-Arg-Val-Leu-COOH and a tyrosine content of less than 3%.

After 7 days, the hair follicle cultures were homogenized and the melanin was extracted at 100° C. for 45 minutes using a 1 M NaOH solution which included 10% by volume DMSO, based on the total volume of the extraction solution. Aliquots of these extracts were then transferred into a 96-well cell culture plate and the extinction or the optical density at a wavelength of 492 nm was measured using a spectral photometer Spektramax plus 384 from the company Molecular Devices (USA). The results were evaluated using the Softmax Pro software from the company Molecular Devices (USA).

Table 2 shows the melanin content of all hair follicle cultures. The melanin content of the untreated hair follicle cultures V serves as a reference value and corresponds to 100%.

| +Sample | Melanin content [%] |
|---|---|
| V | 100 |
| E-I* | 110 |
| E-II* | 114 |
| E-III* | 115 |

*according to the invention

The use of specific oligopeptides leads to a significantly increased synthesis of melanin and thus to a significant stimulation of the natural pigmentation process of hair follicles. Therefore, by using specific oligopeptides, it is possible both to avoid or reduce the graying of the hair and to achieve repigmentation, that is to say to reduce or resolve the graying of the hair.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic, non-therapeutic method for stimulating the natural pigmentation process in cutaneous appendages, including:
applying to the cutaneous appendages at least one oligopeptide which comprises at least one amino acid sequence of formula (I)
(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1, and
the amino group is in non-protonated and/or protonated form and the carboxy groups are in non-deprotonated and/or deprotonated form.

2. The method according to claim 1, wherein applying the at least one oligopeptide stimulates melanogenesis in the cutaneous appendages.

3. The method according to claim 1, wherein the oligopeptide comprises 5 to 15 amino acids.

4. The method according to claim 3, wherein the oligopeptide comprises 8, 9 or 10 amino acids.

5. The method according to claim 1, wherein the oligopeptide has an average molecular weight M_{w} of 400 to 2000 Da.

6. The method according to claim 5, wherein the oligopeptide has an average molecular weight M_{w} of 550 to 1000 Da.

7. The method according to claim 1, wherein in formula (I), B represents the amino acid Tyr, C represents the amino acid lle, D represents the amino acid Arg, E represents the amino acid Val, F represents the amino acid Leu, a represents the integer 0, and b, c, d, e and f each represent the integer 1.

8. The method according to claim 1, wherein in the amino acid sequence of formula (I), A represents the amino acid Leu, B represents the amino acid Tyr, C represents the amino acid lle, D represents the amino acid Arg, E represents the amino acid Val, F represents the amino acid Leu, and a, b, c, d and e each represent the integer 1.

9. The method according to claim 1, wherein at least 0.1% by weight of all oligopeptides have an amino acid sequence of formula (I) in which B represents the amino acid Tyr, C represents the amino acid lle, D represents the amino acid Arg, E represents the amino acid Val and F represents the amino acid Leu, a represents the integer 0, and b, c, d, e and f each represent the integer 1.

10. The method according to claim 9, wherein at least 10% by weight of all oligopeptides have an amino acid sequence of formula (I) in which B represents the amino acid Tyr, C represents the amino acid lle, D represents the amino acid Arg, E represents the amino acid Val and F represents the amino acid Leu, a represents the integer 0, and b, c, d, e and f each represent the integer 1.

11. The method according to claim 1, wherein the oligopeptide is applied in a total amount of 0.001 to 3% by weight based on the total weight of a composition including the at least one oligopeptide, optionally a carrier and optionally further ingredients.

12. A cosmetic, non-therapeutic method for stimulating the natural pigmentation process in cutaneous appendages, including:
applying to the cutaneous appendages a cosmetic agent that includes, in a cosmetically acceptable carrier, at least one oligopeptide comprising at least one amino acid sequence of formula (I)
(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1, the amino group is in non-protonated and/or protonated form and the carboxy groups are in non-deprotonated and/or deprotonated form.

13. A cosmetic, non-therapeutic method for stimulating the natural pigmentation process of cutaneous appendages, in particular the melanogenesis and/or pigmentation of the hair, for preventing and/or reducing the graying of the hair and/or for repigmenting hair that has turned gray, which includes:
a) providing an oligopeptide which includes at least one amino acid sequence of formula (I)
(H)NH-(A)ₐ-(B)_{b}-Glu-Glu-Glu-(C)_{c}-(D)_{d}-(E)_{c}-(F)_{f}-CO—(OH) (I)
in which
A, B, C, D, E and F, each independently of one another, represent the amino acids Leu, Tyr, lle, Arg, Val, Ser, Thr and Asp,
a, b, c, d, e and f, each independently of one another, represent the integers 0 or 1,
the amino group is in non-protonated and/or protonated form and the carboxy groups are in non-deprotonated and/or deprotonated form,
b) topically applying the at least one oligopeptide provided in method step a) to the cutaneous appendages,
c) leaving the at least one oligopeptide applied in method step b) on the cutaneous appendages for a duration of from 30 seconds to 2 hours, and
d) optionally rinsing out the cosmetic agent that was left on the cutaneous appendages in method step c).


SEQUENCE LISTING (7 sequences)

SEQ ID NO: 1 - protein, 3 aa, Artificial
  Peptide
EEE

SEQ ID NO: 2 - protein, 4 aa, Artificial
  Peptide
YEEE

SEQ ID NO: 3 - protein, 4 aa, Artificial
  Peptide
EEEI

SEQ ID NO: 4 - protein, 6 aa, Artificial
  Peptide
YEEEIR

SEQ ID NO: 5 - protein, 7 aa, Artificial
  Peptide
YEEEIRV

SEQ ID NO: 6 - protein, 8 aa, Artificial
  Peptide
YEEEIRVL

SEQ ID NO: 7 - protein, 9 aa, Artificial
  Peptide
LYEEEIRVL